(19) United States
(12) Patent Application Publication
(10) Pub. No.: US 2022/0295025 A1
(43) Pub. Date: Sep. 15, 2022

(54) PROJECTION SYSTEM WITH INTERACTIVE EXCLUSION ZONES AND TOPOLOGICAL ADJUSTMENT

(71) Applicant: Daniel SEIDEL, Columbia, MO (US)

(72) Inventor: Daniel SEIDEL, Columbia, MO (US)

(21) Appl. No.: 17/633,360

(22) PCT Filed: Apr. 13, 2020
(86) PCT No.: PCT/US2020/028017

§ 371 Date: Feb. 7, 2022

Related U.S. Application Data
(60) Provisional application No. 62/920,122, filed on Apr. 12, 2019.

(51) Int. Cl. H04N 9/31; G09F 19/22

(52) U.S. Cl. CPC H04N 9/3185; H04N 9/3194; H04N 9/3155; G09F 19/22

(57) ABSTRACT

Apparatuses, methods, and systems for projecting images into a projection zone are provided, while having the capability to detect the presence and movement of objects in the projection zone and to interact with those objects, according to programmed interactions. One of the programmed interactions is to detect objects in the projection zone and avoid projecting light onto them. The capability to detect and avoid objects in the projection zone allows for the use of high intensity light images including laser light images around people and animals without the risk of eye injury. Another programmed interaction is to project an illuminated image around people and objects in the projection zone to emphasize their presence and movement. Sensed topography data advanced geometry correction for projecting geometrically accurate images onto uneven surfaces. Advanced beam shaping optics enable long distance projections at low angles onto unprepared surfaces.

### CROSS-REFERENCE TO RELATED APPLICATION

This application is related to of U.S. Provisional Application 62/920,122, filed Apr. 12, 2019.

### TECHNICAL FIELD

The present disclosure relates generally to one or more methods, systems, and/or apparatuses for interactively projecting one or more images on a surface, and further includes eye safety features and other interactive capabilities.

### BACKGROUND ART

Presently, there are many types of optical projectors including high intensity laser projectors. High intensity projectors must be operated with precautions to avoid eye damage. Coherent laser light can be especially damaging to eyes and skin. The potential for eye damage has limited the use of high intensity optical projectors.

Presently, there are a few types of projectors that can alter the projected images to react to motions and gestures of the users. For example, U.S. Pat. No. 8,290,208 describes a system for “enhanced safety during laser projection” by attempting to detect an individual's head, define a “head blanking region”, and then track the “head blanking region” to avoid projecting laser light at the individual's head. Most of these projectors are used for entertainment, presentation, and visual aesthetics.

Reactive projectors are not commonly employed in industrial applications. Opportunity exists for a high intensity interactive projector with safety features that allow safe operation around people without risk of eye and skin damage. Opportunity exists for said high intensity interactive projector that suitable for projecting clearly visible, long range, geometrically reliable images onto uneven surfaces in night or daylight conditions.

### SUMMARY DISCLOSURE OF INVENTION

The following presents a simplified summary in order to provide a basic understanding of some aspects of the invention. This summary is not an extensive overview. It is not intended to identify key or critical elements of the invention or to delineate the scope of the invention. The following summary merely presents some concepts of the invention in a simplified form as a prelude to the more detailed description provided below.

Aspects of the present invention relate to optical projectors including laser projectors and projectors having eye safety features and interactive capabilities.

An Interactive Projection System (“IPS”) is capable of projecting light images into a projection zone. The IPS is capable of sensing the projection environment with accuracy in three dimensions. The ability to perceive the projection environment allows advanced geometric correction so that projections are geometrically accurate even on unprepared surfaces. The IPS is also capable of sensing and reacting to the presence and movement of objects within the projection zone according to programmed interactions. One programmed interaction may be to avoid projecting light onto protected objects in the projection zone. Such an ability to sense and avoid protected objects would allow projection of high intensity light such as laser light without the risk of eye damage or skin discomfort to people within the projection zone. Sensed topography data allows the IPS to perform advanced geometry correction and project geometrically accurate images even onto uneven surfaces. IPS has advanced beam shaping optics that enable long distance projections at low angles onto unprepared surfaces.

Aspects of the present invention may include a computerized system for interactively projecting images into a projection zone. An exemplary system may include, but is not limited to, at least one light projecting device, at least one computing device, where the computing device is in operative communication with the at least one light projecting device for transmitting control signals to the at least one light projecting device. The computing device may include, among other things, one or more computer processors. The exemplary system may further include one or more computer-readable storage media having stored thereon computer-processor executable instructions, with the instructions including instructions for controlling the at least one light projecting device to project one or more pre-determined images into the projection zone.

### BRIEF DESCRIPTION OF THE DRAWINGS

For a better understanding of the disclosure, and to show by way of example how the same may be carried into effect, reference is now made to the detailed description along with the accompanying figures in which corresponding numerals in the different figures refer to corresponding parts and in which the drawings show several exemplary embodiments:

FIG. 1 illustrates an exemplary process flow diagram for an IPS, according to various aspects described herein.

FIG. 2 illustrates an exemplary diagram of an IPS, according to various aspects described herein. In this example, the exemplary IPS includes a projector module, control module, and scanner module mounted on a mast.

FIG. 3 illustrates an exemplary diagram of an IPS projecting an image into a projection zone, according to various aspects described herein.

FIG. 4 illustrates an exemplary diagram of various projected signals for automobile traffic control and advisory, according to various aspects described herein.

FIG. 5 illustrates an exemplary diagram of the IPS projecting various signals onto an automobile traffic intersection, according to various aspects described herein.

FIG. 6 illustrates an exemplary diagram of various projected signals for airport traffic control and advisory, according to various aspects described herein.

FIG. 7 illustrates an exemplary diagram of the IPS projecting signals onto airport runways and taxiways, according to various aspects described herein.

FIG. 8 illustrates another exemplary diagram of the IPS projecting signals onto airport runways and taxiways, according to various aspects described herein.

FIG. 9 is a block diagram illustrating an example of a suitable computing system environment in which aspects of the invention may be implemented.

FIG. 10 illustrates an exemplary diagram of the IPS mounted on a train engine projecting graphics onto the railway.

FIG. 11 illustrates an exemplary diagram of the IPS projecting construction reference geometry onto a construction site.

FIG. 12 illustrates an exemplary diagram of the IPS projecting a square onto uneven terrain without geometry correction.

FIG. 13 illustrates an exemplary diagram of the IPS projecting a square onto uneven terrain with geometry correction.

FIG. 14 illustrates an exemplary diagram of the IPS generating a directional photoacoustic effect.

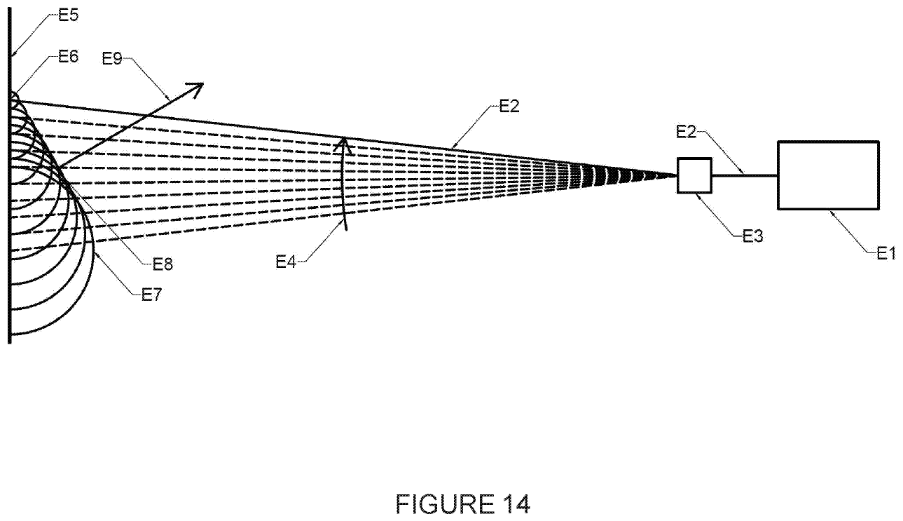

### DETAILED DESCRIPTION OF THE INVENTION

In the following description of the various embodiments, reference is made to the accompanying drawings, which form a part hereof, and in which is shown by way of illustration various embodiments in which features may be practiced. It is to be understood that other embodiments may be utilized, and structural and functional modifications may be made without departing from the scope of the present invention.

As noted above, there are presently many types of optical projectors including high intensity laser projectors. High intensity projectors must be operated with precautions to avoid eye damage. Coherent laser light can be especially damaging to eyes and skin. The potential for eye damage has limited the use of high intensity optical projectors.

Presently, there are a few types of projectors that can alter the projected images to react to motions and gestures of the users. Most of these projectors are used for entertainment, presentation, and visual aesthetics. Reactive projectors are not commonly employed in industrial applications.

Aspects of an exemplary IPS generally contemplate an optical projection system having the capability to detect the presence and movement of objects in the projection zone and to interact with those objects, according to programmed interactions. One of the programmed interactions may be to detect objects in the projection zone and avoid projecting light onto them. The capability to detect and avoid objects in the projection zone may allow for the use of high intensity light images including laser light images around people and animals without the risk of eye injury. Another programmed interaction may be to project an illuminated image around people and objects in the projection zone to emphasize their presence and movement.

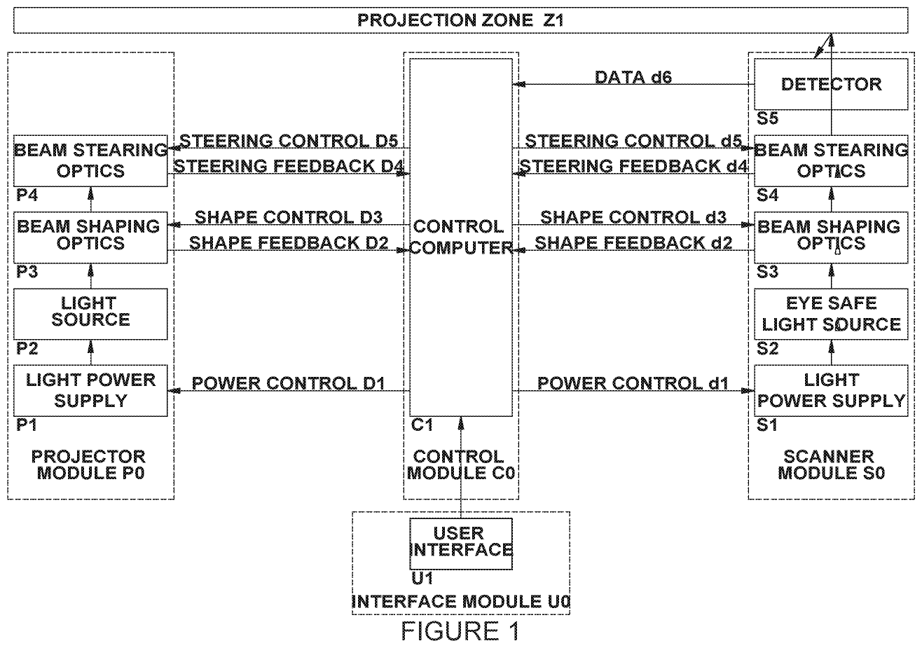

FIG. 1 illustrates an exemplary process flow diagram for an interactive projection system. The example shown in FIG. 1 depicts a projector module P0, a scanner module S0, a control module C0 and an interface module U0 and the various elements within each module. There may be one or more of any elements in the modules. There may be multiple of any module in an IPS system. The modules may be located together in a single unit or remotely located. The signal interactions between modules may be via wire transmission or, wireless transmission. The scanner S0 and projector P0 modules may have one or more processors or controllers that interact with the various elements of the respective modules and communicate with the control computer C1, or the various elements of the respective modules may interact with the control computer C1 directly.

Various projector modules may be configured featuring one or more light sources. By way of demonstration and not limitation, the one or more light sources may include single source, multi-source, incoherent, coherent, laser, visible, invisible, multi-milliwatt, multi-watt, multi kilowatt, or some combination thereof. The beam steering optics may be configured for the desired projection angles including 360-degree projection and global projection. Referring to FIG. 1 and the projector module P0, a light power supply P1 provides electrical power to light source P2. Light source P2 generates a beam of light that is propagated or otherwise directed to the beam shaping optics P3. The beam shaping optics P3 may be actuated via control D3 signals from the control computer C1 to modulate the beam geometry and focus. The shaped beam then propagates to the beam steering optics P4. The beam steering optics P4 may be actuated in relation to control D5 signals from the control computer C1 to direct the light beam to the desired points within the projection zone Z1.

Various scanner modules may be configured to include one or more appropriate scanners, such as but not limited to, passive scanners, active scanners, laser scanners, Light Detection and Ranging (“LIDAR”) scanners, structures light scanners, acoustic scanners, photosensitive scanners, photographic scanners, photogrammetric scanners, video-graphic scanners, Complementary metal-oxide-semiconductor (“CMOS”) scanners, or some combination thereof. Lidar scanners may comprise at least one of, Time of Flight lidar, Continuous Wave Frequency Modulation lidar, Flash lidar, structured light lidar, coherent lidar, incoherent lidar, or any other appropriate lidar. The computer module C1 may be programmed or otherwise configured to analyze data received from the one or more scanners to perform object detection and/or recognition algorithms, e.g., computer vision. Referring to FIG. 1 and the scanner module S0, the scanner module S0 operates similarly to the projector module P0 but with the addition of a detector S5 to sense light reflected from the projection surface. The light source S2 of the scanner module may include visible light, invisible light, or some combination thereof. The light source S2 may be of a magnitude and focus sufficient to cause detectable reflections from the projection zone Z1 at the designed operating distance, but not sufficient to cause eye damage.

The control computer C1 may signal the scanner power supply S1 to produce a pulse of light. The light pulse is modulated through the beam shaping optics S3 directed by the beam steering optics S4 to a point in the projection zone Z1. The pulse may be reflected and/or scattered by a surface in the projection zone Z1. A portion of the pulse may return to the scanner module S0 and be sensed by the detector S5. The control computer C1 may monitor the control and feedback signal d1-d6 data associated with each pulse including a time at which the pulse was generated, one or more modulation settings of the beam shaping optics d2, the position of the beam steering optics d4, a time at which the reflected pulse was detected, other appropriate signals, or some combination thereof. With these values known, the control computer C1 may compute an azimuth and distance to the reflection point and determine the reflective properties of the surface. This process may be performed repeatedly as the pulses are steered to different points in the projection zone. The azimuth, distance, and reflective properties associated with each point may be stored by the control computer C1. In this manner, the projection zone may be scanned, and the data stored as a three-dimensional topographical model of the projection zone Z1.

It should be clear to one of skill in the pertinent arts that various user interface modules U0 may be configured, either computerized or non-computerized, without departing from the scope of the present invention. Furthermore, the IPS may be configured to operate with or without the user interface module U0, without departing from the scope of invention.

Referring again to FIG. 1, the control computer C1 coordinates the power, shape, and direction of the beams propagating from the projector and scanner modules via one or more control and/or feedback signals D1-D5, d1-d6. The control, feedback and/or detector data signals d1-d6 from the scanner module S0 may be computationally analyzed by the control computer C1 to yield topographical data of the projection surface Z1.

Referring further to FIG. 1, operation of an exemplary IPS may generally proceed as follows: The user initiates an IPS setup mode via the user interface U1. The user interface U1 prompts the user to ensure that the projection zone Z1 is void of people or other light sensitive objects. When the user confirms that the projection zone Z1 is clear, the control module C0 and scanner module S0 perform a scan of the projection zone Z1. The scan is stored in the control computer S1 memory as the baseline scan for the projection zone Z1. The control computer C1 presents the baseline image to the user via the user interface U1. The user adds any combination of text, symbols, images, or animations to the baseline image via the user interface U1. When the user initiates projection mode, the control module C0 controls the projector module P0 to trace the graphic images defined by the user onto the projection surface.

The IPS may be programmed with many interactive behaviors. The user may initiate pre-programed interactive behaviors via the user interface U1. The user may also program new interactive behaviors via the user interface U1. These interactive behaviors generally cause at least one associated correction factor to be applied to the image or cause the projector to project the image in an otherwise altered form. These “correction factors” are described herein. One programmed behavior may be to detect objects in the projection zone Z1 and avoid projecting light onto them. Such a “detect and avoid” feature may be accomplished as follows: The scanner module S0 repeatedly scans the projection zone Z1 and the control module C0 compares the current scan with the baseline scan. If any regions of the current scan are different than the baseline scan, the control computer C1 defines that those regions as occupied by a protected object 5 and defines a protection zone 7 with respect to those protected objects. For example, the IPS may find and exclude objects that were not present in the baseline image and/or may utilize more advanced algorithm to identity what the objects are and apply correction factors based on the identity of the objects. These protection zones 7 are hereinafter referred to as protected object zones 7. In some instances, the protected object zone 7 may be larger than an associated protected object 5 by a pre-defined margin of safety. The control computer C1 may monitor the beam steering control or feedback signals D4, D5 from the projector module P0. If a beam from the projector module is preparing to steer into a protected object zone 7, the control computer C1 may apply a “correction factor” to interrupt the power to the light source P2 in the projection module P0 until the beam is steered outside of the protected object zone 7. In this manner, the control computer C1 may disallow projection into any protected object zone 7 on a “real-time” or near “real-time” basis. The resulting effect is that people, animals, or other objects may be present or move into in the projection zone and the IPS will interactively avoid (or attempt to avoid) projecting light onto them.

Another programmed behavior may be to project an illuminated graphic around protected objects 7 to emphasize their presence and movement. Another programmed feature may be geometric correction of projection images. Without adjustment, a projected image will be distorted if the projection surface is not perpendicular to the projection beam, or if the projection surface is not flat. The IPS control module C0 may use topographical data from the scanner module S0 (e.g., azimuth information, other elevation or topographical information) to adjust the projection image for non-perpendicular projection angles and non-flat topography, so that the image will appear as intended or as close as reasonably possible given the uneven projection zone.

Another programmed feature may be spot geometry adjustment. Where a projector beam or scanner beam contacts a projection surface it produces an illuminated spot on the projection surface. The spot geometry depends on the beam geometry and the angle of intercept between the beam and the projection surface. If the beam geometry is constant and the topography of the projection zone varies, the spot geometry will vary throughout the projected image. An IPS control module C0 may use topographical data from the scanner module S0 (and/or user-provided information or other sources of topographical data for the projection zone) to adjust the geometry of the scanner and projector beams via one or more of the beam shaping optics to P3, S3 produce the intended spot geometry throughout the image.

Another programmed feature may be beam attenuation control. The control computer C1 may control one or more aspects of beam divergence and therefore the beam attenuation via the beam shaping optics P3, S3. For example, when one or more beams are projected in a direction where there is no terminating surface, the beam divergence may be adjusted to produce a non-hazardous beam intensity.

Another programmed feature may be brightness adjustment. As described above, the topographical data from the scanner module S0 may include distance, azimuth, and reflective property data associated with various points of the projection zone. The control module may use this data to adjust the beam intensities of the projector P0 and scanner modules S0 to produce the intended brightness throughout the image.

Another programmed feature may be movement correction. Without movement correction, the projected image would be displaced by any movement of the projector. The control module may use one or more elements of the topographical data of the projection zone (such as those described above) to define stationary reference points. The user may add physical reference objects to the projection zone. These reference objects may have specific geometric or reflective properties that make them easily identifiable to the IPS. The scanner module S0 repeatedly measures the distance and azimuth to the reference points. The control module uses this data to repeatedly determine the position of the scanner S0 and projector modules P0. The control computer C1 repeatedly adjusts the projection image data going to the projector module P0 to correct for the movement of the projector module P0. The effect may be that the projected image will remain in the intended location even if the projector module PO is moving.

One or more additional accessory modules may be added to the IPS to add functionality. By way of demonstration and not limitation, such accessory modules may include but are not limited to, a light sensing module (to determine ambient light levels and adjust the projection intensity to achieve the desired contrast ratio), a gravity sensing module (to provide a gravity reference), a gyroscopic sensor module (to provide movement and orientation data), and inertial sensor module (to provide movement and orientation data), a Global Positioning System module (to provide location, orientation and movement data), a remote control module (to provide remote control of the IPS), a network module (to provide networking capabilities), or some combination thereof.

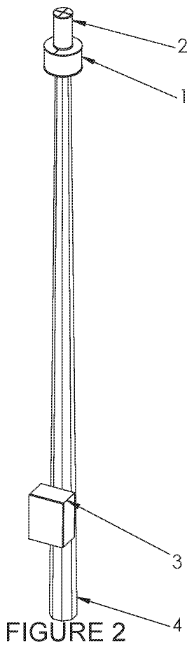

FIG. 2 illustrates an exemplary IPS with the projector module 1, scanner module 2, and control module 3 mounted on a mast 4.

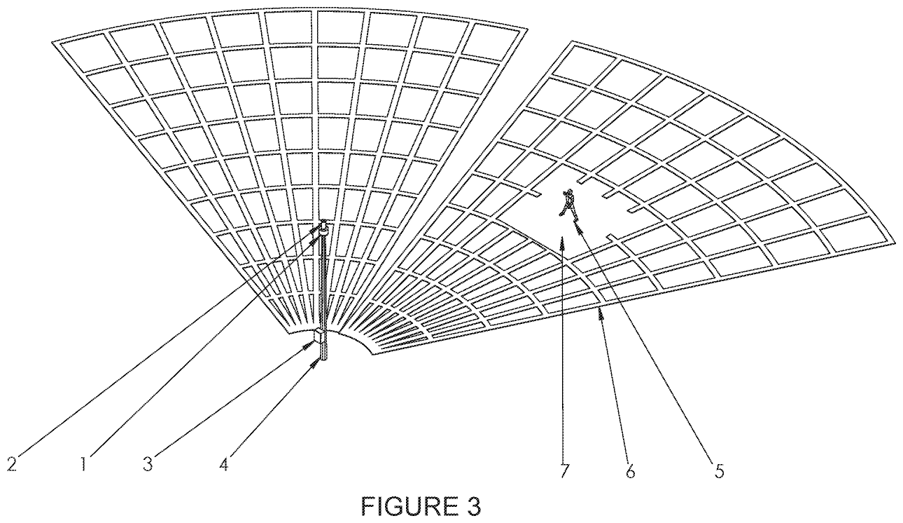

FIG. 3 illustrates an exemplary IPS with the projector module 1, scanner module 2, and control module 3 mounted on a mast 4. The projector module 1 is depicted projecting grid images 6 onto a surface. A protected object zone 7 is depicted surrounding a protected object (person) 5 standing within the projection image 6.

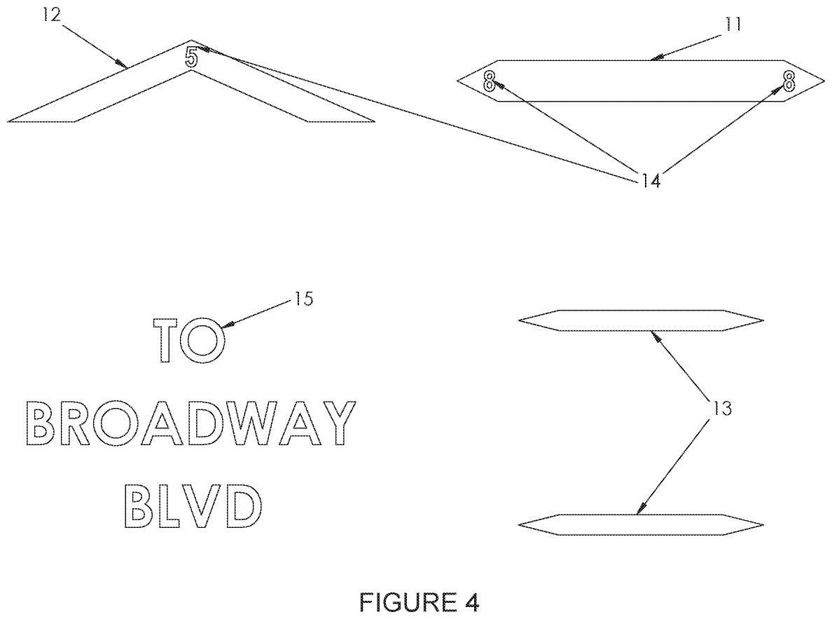

FIG. 4 illustrates examples of various projected signals for automobile traffic control and advisory, e.g., a projected stop signal 11, a project go signal 12 (both of which include a projected countdown to signal changes 14), a projected pedestrian alert 13, and projected advisory information 15.

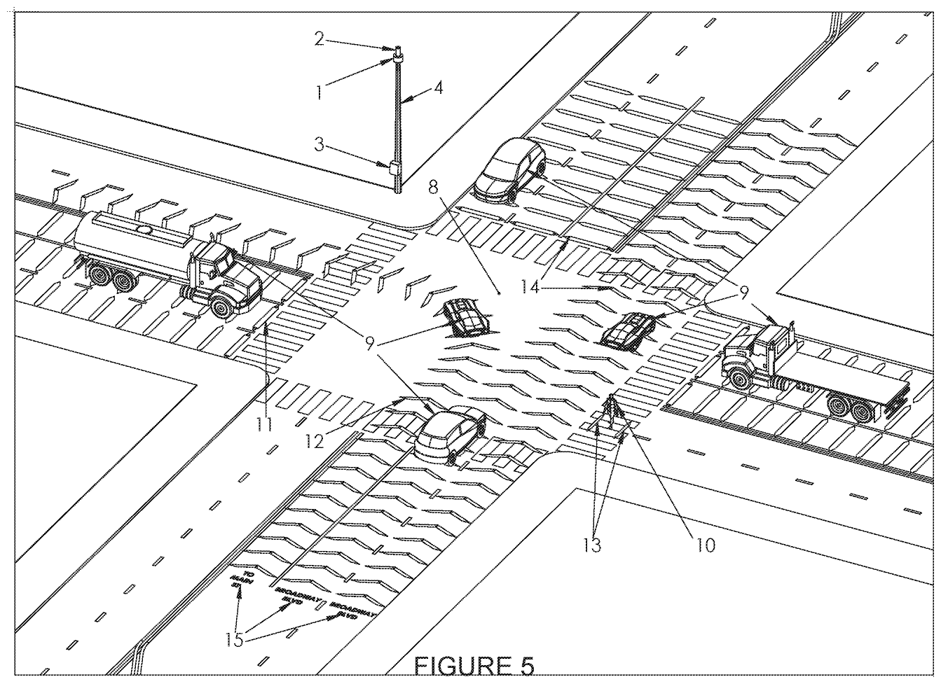

FIG. 5 illustrates an exemplary IPS projecting various signals onto an automobile traffic intersection. For example, FIG. 5 shows the projector module 1, scanner module 2, and control module 3 mounted on a mast 4, a street intersection 8, multiple automobiles 9, a pedestrian 10, a projected stop signal 11, a project go signal 12, a projected pedestrian alert 13, and projected advisory information 15. According to aspects of the present invention, one or more IPS can enhance street traffic control by projecting traffic control signals and information onto streets. IPS can replace or supplement overhead traffic signals. IPS on emergency vehicles or ground structures can project, stop signals, merge signals, lane closure signals, routing signals for normal and emergency operations. IPS can be used as advanced illumination headlights. IPS headlights can project a wide beam to illuminate surroundings. If another vehicle is detected, IPS will make an exclusion zone to avoid projection onto the other vehicle. IPS headlights can detect curvature of the road and steering inputs of the car and adjust the beams to illuminate the appropriate section of roadway. IPS headlights can highlight obstacles such as pedestrians and animals IPS installed at intersections can project signals onto pedestrian crosswalks. Signals can be presented by graphics, text and audio. Examples of signals are: Walk signal, do not walk signal, “clear the walkway” signal, countdown to signal change. Pedestrians will be followed by an exclusion zone and a pedestrian highlight increase their visibility to drivers. If IPS detects a vehicle is violating or about to violate a traffic control signal, recording will be initiated, a projected stop signal will be presented to the vehicle and the vehicles path will be highlighted by a projected warning signal to alert pedestrians and drivers. IPS may additionally be deployed on vehicles or structures to direct vehicle traffic. Various “Go” “Stop” “Merge” symbols and text may be projected to guide traffic around accident scenes, around construction sites, or through detours.

In the context of automobile control and pedestrian/crowd control, one or more IPS may be utilized to project parking stall lines, graphics and text. Lines can be projected only and thereby remain dynamic and changeable. An operator can specify spacing or stall number and the projection will adjust to meet the specifications. Projected lines can be painted to make them permanent. Stalls may be graphically designated as open, reserved, handicapped, permit only, time limited. Designations can be changed manually or automatically by time triggers, occupancy triggers or other programmed parameters. One parking stall may be designated as handicapped. When it becomes occupied, another stall switches its designation to handicapped and adjusts its spacing to meet the requirements for handicapped spaces. Arrows and numbers may be projected to lead drivers to empty parking spaces. Time till parking expiration may also be projected. Projected parking reference works well on paved and unpaved surfaces. Additionally, IPS may project direction signals, and text instructions onto ground, signs, or other surfaces, to direct people to desired areas or dissuade them from prohibited areas. Projected crowd control signals can be used for normal events, or emergency evacuations.

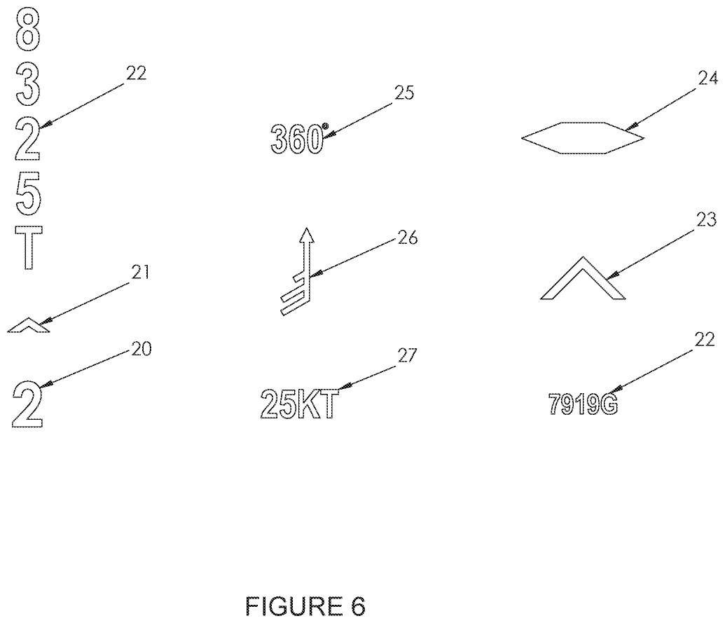

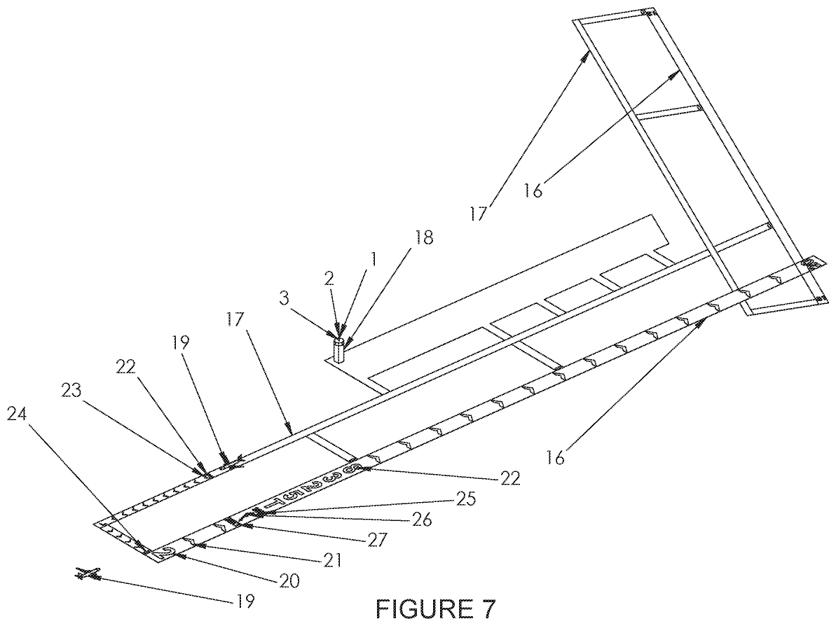

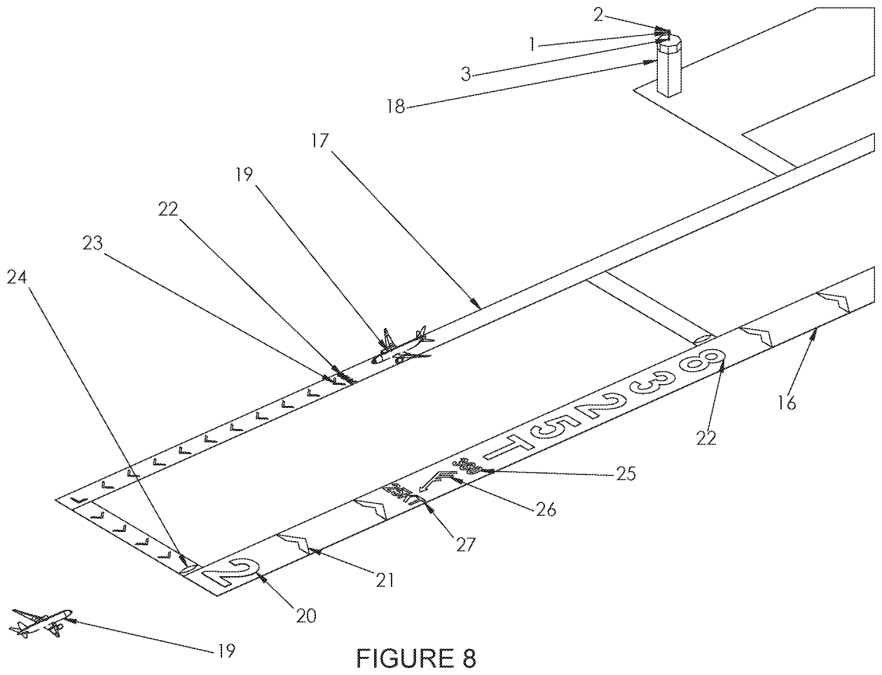

FIG. 6 illustrates exemplary projected signals for airport traffic control and advisory, e.g., a projected runway number 20, a projected clear to land/take-off signal 21, a projected tail number 22, a projected clear to taxi signal 23, a projected stop signal 24, a projected wind direction value 25, a project wind direction/speed symbol 26, and a projected wind speed value 27. FIG. 7 illustrates an exemplary diagram of the IPS projecting the aforementioned signals onto an airport runway 16 and taxiway 17. In this example, an exemplary IPS system (e.g., elements 1, 2, 3) are mounted or otherwise placed on an air traffic control (“ATC”) tower. Advantageously, these lighted projections are more immediately visible to a pilot in an aircraft 19, in comparison to indictors painted on runways and taxiways. FIG. 8 illustrates another exemplary IPS projecting signals onto airport runways and taxiways. In other examples (not shown the FIGURES), the IPS or some portion thereof may be mounted or otherwise affixed to one or more vehicles, such as but not limited to, trains, automobiles, planes, unmanned aerial vehicles/systems, other appropriate vehicles, or some combination thereof.

According to aspects of the present invention, the IPS may comprise one or more modules that can be added to customize functionality. For example, one of modules may comprises a scanner module, where the scanner module uses one or more perception apparatus such as lidar, camera, sonar, radar, or other appropriate method or means to perceive the projection environment and objects therein. In one embodiment, an exemplary IPS utilizes a lidar module in conjunction with a camera module. The lidar module provides accurate topographical data of the projection environment, while an exemplary camera module provides data for object recognition. As computer vision and photogrammetry techniques advance, IPS functions in some embodiments may be accomplished with camera only without the need for lidar.

Another exemplary module may comprise a computer module, where the module receives data from the scanner module, other input modules, or some combination thereof, and controls one or more output modules, such as but not limited to, one or more projector modules to accomplish IPS functions. Other exemplary modules may include a projector module, wherein an exemplary projector module projects luminous graphics and animations into the projection zone. The projector module may selectively use focused light, coherent light, laser light, collimated light, structured light, twisted light, other forms of light, or some combination thereof. The projector may additionally use lenses, mirrors and diffraction gratings to collimate, focus, shape, and structure light. While current projectors use lenses to shape the beam in all dimensions simultaneously, an IPS may utilize lenses, mirrors, diffraction elements, other appropriate methods or means, or some combination thereof, to shape beam dimensions independently. This independent control allows beam shapes to be optimized for long distance projections and low projection angles with minimal divergence and attenuation. To achieve low divergence and favorable diffraction limited spot size, the beam shaping optics may be modulated to produce a beam shape that is sufficiently large at the aperture and focuses down to the desired spot size at the projection surface. One current problem with long distance, low angle projections is inconsistent spot dimensions that result from the variation in the angle of intercept between near and far field projection. According to aspects of the present invention, this problem may be overcome by modulating separate optical elements to individually control the spot dimensions. One embodiment of the projector optics comprises the laser source, a collimating lens, a focal lens that may be actuated to vary the X dimension of the beam shape, a focal lens that may be actuated to vary the Y dimension of the beam shape, a beam steering lens that may be actuated to modulate the beam path. Alternatively, prisms may be used to modulate the beam, shape and beam steering mirrors may be used to modulate the beam path. After the beam steering optics, optics may be added to expand or narrow the projection field. A wide-angle lens can provide a hemispherical projection field. A spherical reflector can provide a near spherical projection field. One or more prisms may be used to narrow the projection field in the Y dimension to compensate for low projection angles. The projector module can project onto surfaces or into space using volumetric projections and holography techniques. One such holography technique is to use focused light or other radiant energy to heat air or other medium. The heated medium creates a luminous plasma pixel at the desired location. Multiple luminous pixels are arranged into a volumetric holographic image.

Another exemplary module may comprise a gravity reference module, wherein the module may utilize levels, accelerometers, or other gravity sensing hardware, or some combination thereof, to determine orientation of the IPS relative to the direction of gravity. Other exemplary modules may include: a geo-reference module that utilizes Geo Positioning System (“GPS”), Global Navigation Satellite System (“GNSS”), or other suitable geo-positioning hardware and software, or some combination thereof, to determine geographical location, orientation and movement of the IPS; an inertia model that utilizes inertia sensing hardware and software, such as inertial navigation system (“INS”), inertial measurement unit (“IMU”) to determine movement, position, orientation of the IPS; a sound module that utilizes microphones, speakers, phased arrays of microphones, phased arrays of speakers, photoacoustic transmitters, photoacoustic microphones, other suitable devices, or some combination thereof, to sense and project sound for, communication applications, cymatic applications, and industrial applications.

With respect to the various IPS functions, an exemplary IP may utilize the information received from the various modules to interpret the information using various computing techniques, wherein commands are executed to accomplish various programmed functions and interactions. For example, an exemplary function may comprise a calibration function, a function that checks one or more position, orientation, and/or alignment of various hardware elements, and thereafter recommending or suggesting calibration action to be taken manually by a user or performed automatically. For example, a projector module may project one or more points onto a surface that correspond with calibration points being monitored by the scanner module. If the projected dots align with the scanned calibration points, calibration is verified. If there is deviation between the calibration points and the projected points, the deviation values may be presented for adjustment. Software adjustments may be made on command or automatically. Hardware adjustments may be made manually or mechanized for automatic calibration.

Another exemplary function may comprise a scanning function, wherein a scanning function may operate to scan the projection environment to perceive topographical data including, but not limited to, geography, geometry, illumination, and/or reflectivity. Scan data may be streamed to a computer module, where the information may be analyzed and used to accomplish the various IPS functions. One embodiment of scan data is a point cloud model of the scan environment wherein each point contains property information comprising location coordinates, signal strength, reflectivity, ambient illumination, motion vectors.

Another exemplary function comprises a perception function, wherein an IPS computer module analyzes scan data using any combination of computer perception techniques. Examples of computing techniques include, but are not limited to, Simultaneous Localization And Mapping (“SLAM”), background subtraction, edge detection, computer vision, photogrammetry, structured light, deep learning, neural networks, canny edge detection, Hough transform, artificial intelligence, augmented reality, Computer Vision, Stereo Vision, Monocular Depth Estimation, Parallax, Triangulation. The perception data may be utilized to construct a three-dimensional model of the projection environment and to accomplish the other IPS functions.

Other functions may include, but are not limited to, an object detection function to detect the position, size, orientation, and movement of objects in the projection zone, an object identification function that utilizes one or more perception techniques to detect the position, size, orientation, and movement of objects in the projection zone, an object exclusion function wherein data describing the position, size, orientation, and movement of objects in the projection zone is used to establish exclusion zones around protected objects. The projection is altered to prohibit projection into the exclusion zones. This feature allows people and animals to interact in proximity of the high-powered projections without risk of eye or skin damage. Additionally, an object highlight function wherein data describing the position, size, orientation, and movement of objects in the projection zone is used to establish highlight graphics on or around objects of interest. This feature very effectively draws attention to objects of interest with direct illumination and or proximity graphics.

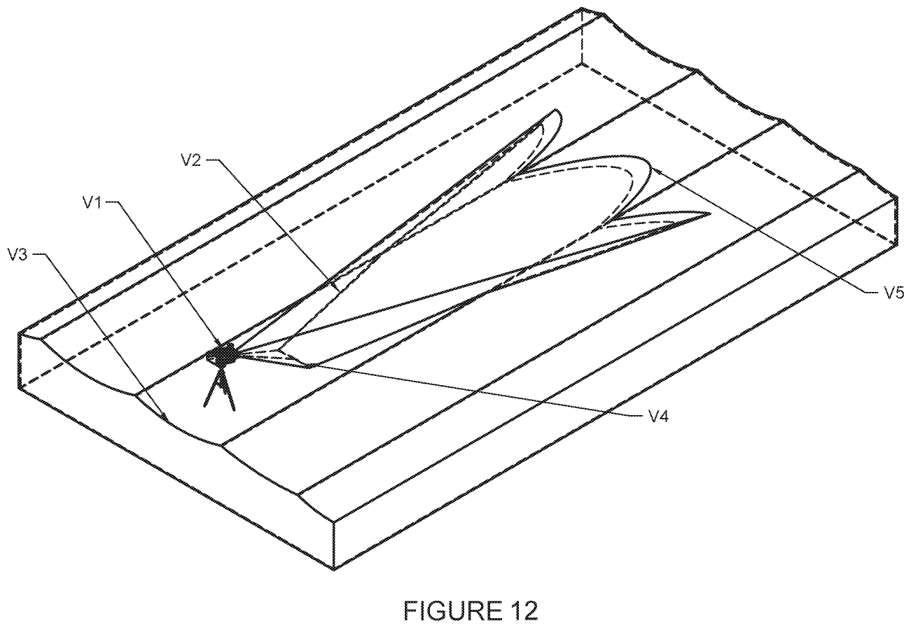

Other functions allow for geometry detection where data from the scanner module is analyzed by the computer module using various computing techniques to compute the topographical properties of the projection zone and objects in the projection zone, e.g., contours, surfaces, edges, slopes, reflectivity, and geometry correction where the scanner module scans the topography of the projection environment and adjusts the projected image to display with the intended geometry. This feature allows long-distance, geometrically accurate projections onto complex topography and objects with complex shapes. For example, a projection image may be selected, each point of the image having X and Y coordinates relative to an origin in a cartesian coordinate system. The user assigns the origin of the projected image to a desired location on the site and chooses the geo-correct command The projector orientation may be determined either by user input or by a gravity sensing module. The position and orientation of the scanner module should be known or otherwise determined from calibration. Using a vector transformation, cartesian coordinates of the scan data are transformed from the optical origin of the scanner module to the optical origin of the projector module. Cartesian coordinates from the projection image are transformed from the image origin to the optical origin of the projector module. For each X,Y,Z coordinate of the projection image, a corresponding X,Y,Z coordinate from the scan data is determined and stored as the geo-corrected image. Instructions are derived to drive the beam steering optics to trace the geo-corrected image. Instructions are derived to drive the beam shaping optics to modulate beam dimensions for consistent line width in both near field and far field projections. As well as location properties, scan data also contains reflective properties of the various scanned surfaces and values for ambient light conditions. Instructions may be derived to modulate beam power and beam shaping optics in relation to the properties of the various projection surfaces. To achieve consistent image brightness, beam power may be increased and concentrated for diffuse surfaces of lower reflectivity and decreased and dispersed for more specular reflective surfaces. If highly specularly reflective surfaces are detected, beam power can be interrupted to exclude those surfaces and avoid stray reflections. Beam power and concentration may also be modulated based on the identification of detected objects. For example, if IPS detects a person in the projection zone, beam speed, power, and concentration may be modulated for the related portions of the projection to not exceed permissible exposure limits for eyes, skin and materials. Beam power and concentration may also be modulated based on the sensed ambient light to enable clear visibility of the projected images across a range from zero ambient light to full daylight conditions. Referring to FIG. 12, an IPS V1 projects the image of a square V2 onto uneven terrain V3 without geometry correction. The image is distorted by the low projection angle and by the varying topography. The far field line width V5 is thickened compared to the near field line width V4 due to the lower angle of intercept at the far field.

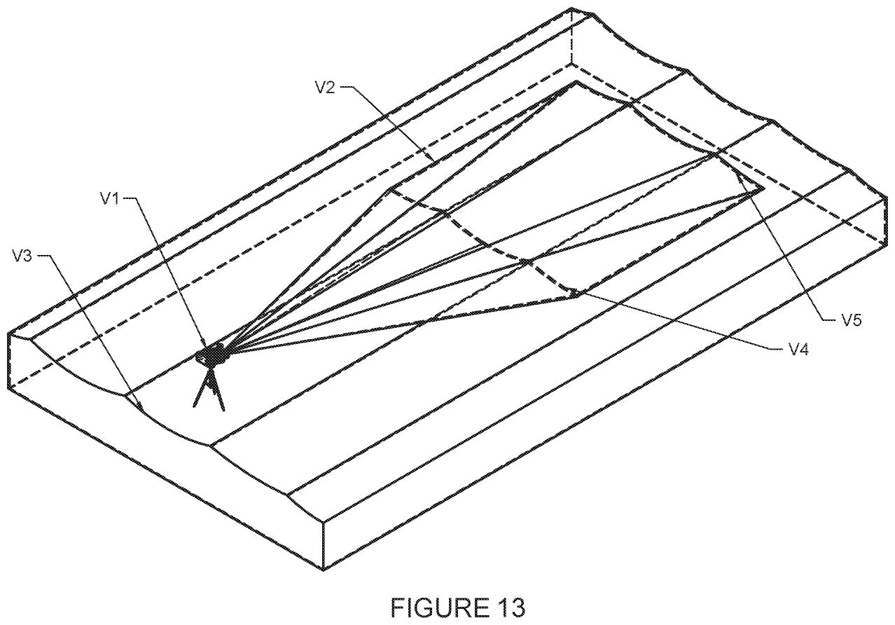

Referring to FIG. 13, an IPS V1 projects the image of a square V2 onto uneven terrain V3 with geometry correction. The projection is mapped to the surface and displays true geometry on the uneven terrain. Beam shaping optics are modulated so that the far field line width V5 is consistent with the near field line width V4 due to the lower angle of intercept at the far field.

According to aspects of the present invention, two-dimensional and three-dimensional geo-referenced data from the scanner module is acquired throughout the process. This as-built data can be transmitted for remote inspection and stored for future reference. Inspectors can review the three-dimensional construction timeline as a video or images that can be rotated and navigated. The time stamped geo-referenced data points allow point to point measurements, slope measurements, geometry verification, and other inspection aids.

In some embodiments, advanced measurements may be acquired, where IPS scan data may be presented on the user interface as a three-dimensional point cloud or mesh. Users can select various points on the point cloud and be presented with measurements relating to the selected points. One IPS accessory is a pointer with reflective or emissive features that make it easily identifiable to IPS scanner modules. Users may use the pointer to expediently select features of the projections or features of the physical projection environment. As the feature selections are detected by the IPS scanner module, highlights are projected onto the features along with measurements associated with those features. Examples of measurements are X component distances, Y component distances, Z component distances, straight line distances, path distances, angle measurements, curvature measurements, area measurements and volume measurements. These measurements are easily derived even over complex topography and geometry that would make current methods inadequate. This method of advanced measurement and on-site display offers clear advantages of expedience and accuracy over current methods of measuring wheels, measuring tapes, range finders, and current survey tools.

Advantageously, an exemplary IPS may bridge the gap between computer aided design and the physical environment (“CAD-to-reality”). CAD can originate in a computer model and be projected onto the environment; or geometry can originate by interacting with projections in the environment. Interacting with the environment will update CAD models. Interacting with CAD models will update projections in the environment. Additionally, perception data may be recorded or stored as desired. Recordings may be continuous, on command, on interval, motion activated, or some combination thereof. Perception data may be presented as a three-dimensional model that can be rotated and navigated. The model may comprise a still model, an animated model, or some combination thereof. Software tools may additionally allow measurements to be made of any features in the model for inspection and verification.

According to aspects of the present invention, an exemplary IPS may be utilized in a number of similar or dissimilar contexts. One or more IPS may be mounted to ground structures, land craft, watercraft, aircraft, and spacecraft, such as masts, towers, buildings, trees, cars, trucks, boats, ships, trains, helicopters, airplanes, satellites. Additionally, each IPS may function alone or be networked with other IPS. For example, one or more IPS may be utilized for animal control. In this example, data from a scanner module is analyzed by a computer module using various computing techniques to identify animals and generate one or more deterrent graphics to be projected by a projector module. Deterrent graphics may utilize a combination of direct illumination, surrounding graphics, intercepting graphics. Deterrent graphics may utilize intensities, colors, geometry, movement, strobing, properties that are psychologically deterring to general or specific animal species, or some combination thereof. In another example, one or more IPS may project beams or images that are attractant to one or more insect species. When IPS detects the presence of an insect and confirms the absence of a human, the beam steering, focus and power are modulated momentarily to deliver a lethal dose of radiant energy to the insect. Insect barriers may be projected to protect a space from insect incursion. One or more IPS may be utilized for intruder detection and deterrent. In this example, data from the scanner module is analyzed by the computer module using various computing techniques to identify intruders and generate deterrent graphics to be projected by the projector module. Deterrent graphics may use a combination of direct illumination, surrounding graphics, intercepting graphics. Deterrent graphics may utilize intensities, colors, geometry, movement, strobing, properties that are psychologically deterring.

One or more IPS may be utilized to display holographic projections. The beam shaping optics of IPS enable volumetric projections or holographic projections. Due to its ability to quickly modulate beam direction, power, and focal point, one or more IPS may be utilized to produce an array of bright pixels that form a volumetric shape. With a sufficient optical power, the one or more projectors may heat the focal points to create an array of plasma pixels. Utilizing the object detection and recognition features of IPS, the holographic projections may interact with people and objects in the projection zone. The directional photoacoustic effect described in this document may be utilized to produce holographic projections with directional or omnidirectional speech, music, or other sounds, or some combination thereof.

One or more IPS may additionally be utilized for aircraft operations. One or more IPS may be stationed on structures such as control towers, beacon towers, lighting masts, or other suitable surfaces, or some combination thereof. According to aspects of the present invention, runway markings may be projected, existing runway markings may be illuminated, or airport identification may be projected onto the surface of the airport or as a holographic text or image above the airport. Additionally, visual glideslope graphics may be projected onto the surface or in space to guide approaching aircraft, an airport beacon signal that portrays airport identification may be projected selectively into the sky and not the ground, airport identification may be portrayed by projected text, shape, color, or flash sequence, air traffic control signals may be projected onto runways and taxiways including tail numbers, directional signals, clearance signals, clearance text instructions. Furthermore, helicopter landing zone graphics may be projected from ground structures, vehicles, or aircraft onto paved surfaces, unpaved surfaces, airports, landing zones, ship decks and such.

In some embodiments, one or more IPS may be equipped with a weather module or otherwise receive and project near real time weather information graphics onto aircraft operation areas. The weather module may use traditional sensors or derive weather information from optical techniques. Examples of weather information may include, but is not limited to, wind speed and direction, altitude, pressure altitude, density altitude, barometric pressure, cloud base height, cloud top height, hazardous weather alerts. Examples of optical techniques for weather sensing may include sensing beam attenuation to determine visibility and other atmospheric properties, sensing beam changes caused by moving atmospheric particles to detect speed and direction of wind and precipitation, sensing beam surface reflectivity changes to detect precipitation type and amount, optical sensors to detect intensity and direction of celestial, atmospheric, and man-made illumination. IPS may optically detect lightning strikes and acoustically detect thunder and present azimuth, range, and intensity information. IPS may adjust beam shape and intensity to adjust for changes in illumination, reflectivity, and visibility. IPS may detect and highlight areas of snow, ice, water and sand to alert pilots and guide plows and other surface treatment measures.

According to aspects of the present invention, one or more IPS may prevent potential runway incursions by monitoring movement of vehicles and aircraft and projecting graphical alerts if a potential conflict is detected. If an incursion occurs, the obstruction may be highlighted to alert other traffic as to the position and movement of the obstruction. IPS may additionally be utilized on aircraft. Structured light may be projected along flight path for increased visibility and collision avoidance, obstacles may be detected and highlighted including powerlines, trees and other obstructions, and landing zone graphics and properties can be projected, such as terrain slope, and wind direction.

According to aspects of the present invention, one or more IPS may be utilized for railway operations. IPS may be stationed on ground structures or trains. For example, warning signals may be projected on the railway ahead of a train to alert drivers, pedestrians and animals of the approaching train, warning signals may also be projected into space ahead of the train using holography techniques. Additionally, animal detection and deterrent graphics may be projected to clear the track of animals IPS may adjust the projected image to match the curvature of tracks, roadways and markings. Another programmed interaction is exclusion zones. If IPS identifies protected objects in the projection zone, it will establish exclusion zones around the protected objects. No laser projection will be allowed into the exclusion zones. The exclusion zone feature ensures eye safety for people and animals in the projection zone. IPS will determine the size and position of objects in the projection zone. A highlight may be projected around selected objects. Another programmed interaction is animal deterrent. Various graphics may be projected with color, intensity, movement and strobing behaviors to discourage animals from entering selected areas. IPS can detect problems in the railway and create a record of the problem and location. Such problems may include, but are not limited to, track deviations, track displacement, thermal expansion, vegetation encroachment, damaged rails, damaged ties, damaged crossings, damaged bridges, ground heave, erosion obstructions. Examples of obstructions may include, but are not limited to, landslides, fallen trees, avalanches, glaciers, vehicles, people, animals IPS may compare data from a scanner module to previously recorded data and identify the train's position. IPS may interpret data from one or more of a scanner module, inertia module, or navigation module to derive the train's speed. IPS may provide estimated time of arrival to selected points, as well as visual and audio collision warnings, e.g., time-until-impact warning. IPS may also project numbers onto crossings indicating time until the train crosses that point. If a possible collision is detected, the obstruction will be highlighted by the projector module, and audiovisual warnings may be displayed to alert the conductor. An audiovisual countdown of time to impact may be presented to the conductor, and a visual countdown of time-to-impact may be projected onto the railway near the obstruction. IPS may be integrated to sound the train whistle automatically when a possible obstruction is detected.

Advantageously, one or more IPS may discern the railway environment and use analytic techniques to document critical, noncritical, or future critical characteristics. Examples of critical characteristics may include, but are not limited to, objects obstructing the track or damaged sections of track. Examples of non-critical characteristics may include, but are not limited to, vegetation growing in the track or objects near but not obstructing the track. Examples of future critical characteristics include, but are not limited to, vegetation growing toward track, trees likely to fall onto track, ground displacement, or track displacement.

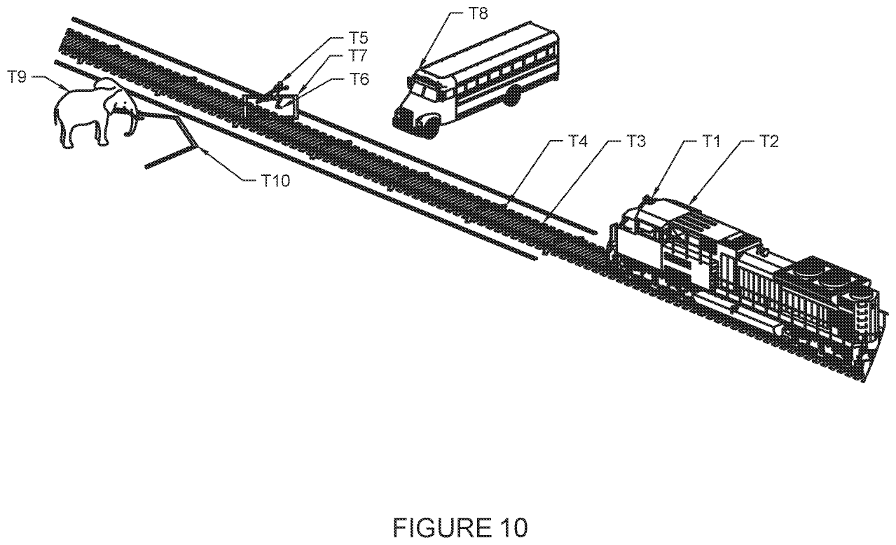

FIG. 10 illustrates an exemplary IPS projecting various signals from a train onto a railway. Referring to FIG. 10, an IPS T1 mounted on a train T2 engine. The IPS T1 projects a luminous “clear the track” signal T4 onto the railway track T3. The “clear the track” signal T4 will be designed to call awareness to the approaching train T2 and thereby prevent accidents due to inattention or low visibility. The “clear the track” signal T4 can be programmed to move, or to be stationary relative to the track T3. A “clear the track” signal T4 that moves along the track T3 at the same speed as the train T2 will allow observers to perceive the direction and speed of the approaching train T2. The “clear the track” signal T4 can also indicate the clearance distance from the track at which a vehicle T8, pedestrian T5, or animal T9 is safe. If an object such as a pedestrian T5, vehicle T8, or animal T9, enters the railway it will be followed by an exclusion zone T6 and an object highlight T7. If an animal is detected approaching the railway, an animal deterrent graphic T10 will be projected between an animal T9 and the railway track T3.

In another embodiment, one or more may be used to aid placement and alignment of objects such as equipment and furniture. For example, IPS can scan a venue and project seating reference lines onto the ground. The seating arrangement can be optimized by desired parameters such as spacing, fire codes, occupancy. When a final arrangement is selected, seats are placed on the reference lines with no manual measuring or marking required. For another example, IPS can be used to guide the placement of loads being moved by cranes, forklifts, and aircraft.

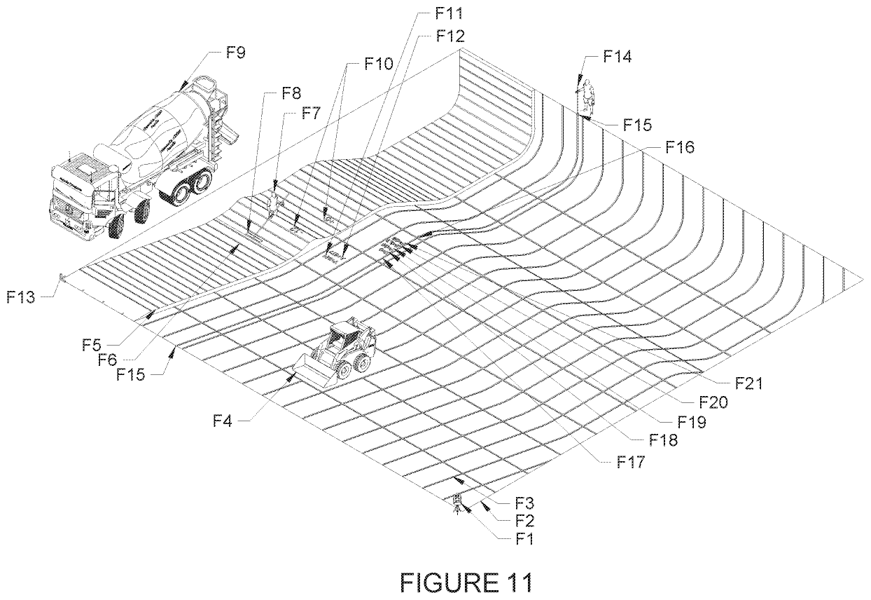

In another embodiment, one or more IPS may enhance construction operations by providing active geometry reference, geography reference, project documentation, project inspection data. An exemplary is illustrated in FIG. 11 and described further herein. In this example, data from one or more scanner modules, GPS module, gravity reference module, other relevant modules, or some combination thereof, is analyzed by a computer module to determine the position and orientation of the IPS, and the geometric properties of topography and objects in the projection zone. IPS geometric correction feature makes it useful for projecting reference graphics that are geometrically accurate. IPS may utilize topographical data acquired by the scanner module to adjust the projected graphics to display as intended even, onto complex topography and at various projection angles. Examples of reference graphics include, but are not limited to, points, lines, arrays, arcs, circles, topographic lines, iso lines, contour lines, isogonic lines, cut lines, fold lines, etch lines level lines, plumb lines, symbols, text, and numerals. These reference graphics may be updated rapidly to provide an active reference that reacts to changes.

An exemplary embodiment of interactive construction reference is described herein. One or more IPS may be set up at a construction site and mounted to a tripod, structure, vehicle, aircraft, person or robot, wherein the IPS scans the topography of the construction site and presents the scanned geometry to a user via the user interface. The user may add construction reference geometry via the user interface. The user may also add construction geometry by placing retroreflective objects or illuminated objects on the construction site. Geometry may also be added by tapping points or tracing lines on the site with a retroreflective or illuminated staff. Commands may be given to the IPS via keyboard, touchscreen, voice commands, gesture commands, or by interacting with command options projected on the site.

If CAD (Computer Aided Design) files for the construction project are available, they may be loaded to the IPS. The user places and orients the CAD geometry over the scanned geometry. If the CAD geometry contains georeferenced coordinates, it can be placed and oriented to the site automatically. The user selects which CAD geometry to project on the site. While inactive projections are generally skewed by uneven terrain and low projection angles, IPS uses position, orientation, and topography data to project accurate geometry onto the site. IPS may compare current scans to construction models and calculate differences in volume and topography. According to aspects of the present invention, one or more IPS project an image of the outline of the foundation onto the construction site and workers are able to see the foundation outline on the construction site visually without the need to receiving/viewing equipment.

As such, workers may begin excavating the foundation based on the projected markings/image. The one or more IPS projects a color-coded active reference grid onto the excavation site. For example, sections of the grid that are below target are projected with yellow, while sections of the grid that are above target are projected with red. In this exemplary embodiment, sections of the grid that are on target are projected with green. If a single color IPS is used, various line types (solid, dashed, dotted) or thicknesses may be utilized to signify deviation in lieu of color. Numbers and symbols may also be projected to signify the amount and direction of deviation from target geometry. Projected volume deviation numbers may indicate how much concrete or other material needs to be added or removed.

Once the foundation is excavated, another reference grid may be projected to indicate where reinforcement bars and hardware should be placed. Workers may quickly place the bars as indicated by the reference grid with no need for measuring and marking. IPS may additionally project lines showing where to place floor drains and other plumbing. Another reference grid may guide the pouring of the concrete foundation. The grid is set up to slope toward the centerline with a concave area around the floor drain so that the foundation sheds water toward the drain. The grid may then appear on the concrete being poured. Some sectors may show in red and show deviation numbers, like −7, indicating that point is too high and needs to be adjusted downward seven centimeters. Some sectors may display in yellow and show numbers like +8 indicating that point is too low and needs to be filled in 8 centimeters. The concrete is worked until all sections are green and deviation numbers are within acceptable limits.

One or more IPS may project an array of dots to show where to place anchor bolts and other relevant hardware. Active reference geometry is projected to guide earth work, masonry work, woodwork, sheetrock work, siding work, shingling work carpet work, painting work, other aspects of construction work, or some combination thereof. Dots, lines, arrays, contours and grids may be projected to align blocks, bricks, mortar, wood beams, wood sheets, metal beams, metal, sheets, siding, shingles, nails, screws, fasteners wood rails, metal rails, ties, earth, gravel, sand, concrete, asphalt, stone, bricks, tiles.

According to aspect of the present invention, one or more IPS may project CAD geometry, scanned geometry, or manually input geometry onto building and finishing materials. For example, the as-built floor plan can be scanned from the site. Carpet is rolled out in an open space, wherein the IPS projects the cut lines onto the carpet. The workers may then cut the carpet with no need for measuring. One or more IPS with sufficient laser power may scorch-mark reference geometry, or even laser cut the construction and finishing materials. These advanced marking and cutting features will advantageously save countless man-hours and eliminate many errors.

Many modern construction projects are designed in a CAD (Computer Aided Design) program. CAD models can be uploaded to a geography reference environment such as google earth, or geo-reference coordinates assigned to the CAD geometry. IPS can use a combination of Global Positioning System (GPS), gravitational, inertial modules to understand its global position and orientation. CAD models with geo-reference coordinates may uploaded to IPS. When coordinates are within the field of view the IPS will project the CAD geometry onto the site according to the coordinates assigned to the various points. The projected geometry would remain stationary even if IPS is moved. For example, consider a road alteration scenario wherein an operator could remotely add geometry to a geo-referenced CAD model to instruct workers to cut a section of pavement from a road to install a culvert. The workers drive a truck equipped with IPS along the specified road. As the truck nears the site the IPS begins projecting the geo-referenced geometry onto the pavement. Workers make the cuts along the projected reference lines and excavate to the depth indicated by the active projected reference grid. Text instructions can also be projected onto the site. IPS visually indicates the position and slope of the culvert pipe, the level of backfill and the contours of the concrete and asphalt and the outlines of markings to be painted.

IPS is also well suited for project documentation. Two-dimensional and three-dimensional geo-referenced data from the scanner module may be acquired throughout the process. This as-built data can be transmitted for remote inspection and stored for future reference. Inspectors can review the three-dimensional construction timeline as a video or images that can be rotated and navigated. The time stamped geo-referenced data points allow point to point measurements, slope measurements, geometry verification, and other inspection aids. A miniaturized version of IPS could be as portable as a hand-held flashlight or lamp. When the IPS is directed toward a surface that has programmed geometry or graphics it will display those graphics onto the surface. It would effectively be augmented reality with no screens, goggles, or other receiver equipment required. IPS can be ruggedized to withstand heat, cold, immersion, pressure, shock, and vibration.

Additionally, IPS is well suited for water and land-based construction projects. Consider the scenario of a bridge constructed over a body of water, wherein one or more IPS may project onto any surface including water. Inertial and geospatial modules allow IPS to understand its position and orientation and to project steady images even if the system is in motion. An IPS set up on shore projects reference marks onto the surface of the water for the placement of pylons. The IPS monitors and adjusts for waves on the surface so the geometry and position of the projection remains accurate. To make the projection more visible, a screen can be floated on the surface to better display the projected geometry. A barge with construction equipment and an IPS approaches the image on the surface. As the barge moves into position the on board IPS begins projecting geo-referenced geometry. The projected geometry is used to position and anchor the barge with the drilling equipment directly over the designated site for the pylon. A waterproof IPS on the bottom of the barge may project reference geometry through the water onto the floor to aid in the precise positioning of tools, equipment, and structures. Structures are placed and concrete is poured with visual reference below and above water. Active visual reference of level, plumb, square, grade, and alignment, greatly improve the speed and accuracy of the construction process. Real-time automated and manual inspection of as-built scan data eliminates errors and provides a detailed record of construction.

As noted above, FIG. 11 illustrates an exemplary IPS projecting various reference graphics for construction of a pool with complex geometry. An IPS F1 set up on a tripod projects the pool outline F2 and excavation reference grid F3 onto the construction site. As a worker with an excavation machine F4 excavates and shapes the site, the IPS F1 scans the new topography and updates the colors of excavation reference grid F3 and the deviation numbers F10 to indicate areas that are high, low, or on target. The worker with an excavation machine F4 excavates and shapes the site until all sections of the excavation reference grid F3 are green and deviation numbers F10 are within acceptable limits. A concrete truck F9 pours concrete F5 into the site. The IPS F1 projects concrete reference contours F6 onto the concrete F5. A worker F7 with a concrete tool F8 works the concrete into the desired shape according to the concrete reference contours F6. The IPS F1 scans and calculates the difference between the scanned volume and the planned volume and projects a volume deviation number F11 onto the site. The volume deviation number F11 indicates how much more concrete is needed to finish the pour. Likewise, the area deviation number F12 indicates how much area remains to be covered. Reference objects F13 may be added to the site. A reference object F13 may have reflective, emissive, and geometric properties that make their position and orientation distinguishable by the IPS F1. An example of a reference object F13 comprises a trident of three arms joined at a vertex and perpendicular to each other. The trident may have retroreflectors and light emitting diodes on the arms and vertex of the trident. A reference object F13 may be used to establish the position and orientation of the origin and axes of a coordinate system or projection. A command staff F14 may be used to give remote commands to the IPS F1. A command staff F14 may have reflective, emissive, and geometric properties that make its position and orientation distinguishable by the IPS. An example of a command staff F14 is a staff with a narrow emissive tip, and a laser that projects a beam from the narrow tip. The user may pinpoint features physically with the narrow tip or optically with the projected laser dot. The user may point the command staff and pulse the laser in a prearranged sequence. The IPS F1 recognizes the pulsed sequence and projects a command menu on the ground where the command staff was pointing. The user may select a command by pulsing the desired command with the laser pointer. A user may select the measure command and select points or features from the projection environment. Selected points F15 and features are highlighted by the IPS F1. F17 indicates a user selected contour. The contour and measurements associated with the contour are projected onto the site. The X measurement F18, Y measurement F19, and Z measurement F20, show the component distances of the contour. The path distance F21 shows the distance along the path of the contour. Other geometric features may be highlighted such as inflection points F16 isolines, and watershed contours. The IPS F1 avoids projecting onto the workers and equipment. IPS may be configured to recognize movements and gestures of the human body, the command staff, the projected laser dot of the command staff, and other objects. IPS responds to the movement and gestures according to programmed interactions. For example, the user activates the laser pointer on the command staff and moves the projected laser dot in a roughly square shape. The IPS projects a square at the corresponding location. The user selects points on the square with the command staff and alters the position and size of the square.

In other embodiments, one or more IPS can guide mining and tunneling operations by using the same projection features described in the construction operations. One or more IPS may be utilized to guide dredging operations. Guidelines, active reference grids, deviation indicators and any other useful data may be projected onto boat surfaces, water surface, or under water terrain. Furthermore, one or more IPS may be utilized to guide search and rescue operations on land, water, or underwater. Search patterns may be projected. Or paths may be projected to guide lost people to extraction points.

In some embodiments, one or more IPS using ultraviolet wavelengths may be used to sterilize surfaces and spaces. The beam steering optics and beam shaping optics can scan spaces and surfaces with ultraviolet (“UV”) beams of sufficient intensity to neutralize pathogens. The scanner module will detect people and prohibit or limit UV exposure to avoid eye or skin damage. IPS can project curtains and enclosures of UV light as a barrier to pathogens. Such UV enclosures can be used to isolate patients especially in hospital overflow situations. Certain high touch surfaces may be specifically designed to be easily sanitized by UV light. For example, door handles and faucets may be constructed of translucent materials to allow penetration and distribution of UV light.

In another embodiment, one or more IPS may be utilized to visually represent sound. Sound modules may be incorporated. Examples of sound modules are microphones, speakers, photoacoustic surfaces. Signals and data from the sound modules will be analyzed by the computer module and used to control visualizations projected by the projector module. Visualizations will visually represent sound properties such as volume, pitch, tone, direction and speed.

If the relative position of the sound source is known, and the topography of the projection zone known, sound visualizations can be made to move with the same speed and direction as the sound. There are several methods to determine the relative position of the sound source. The coordinates of sound sources can be measured and entered manually. A sound module with an array of microphones can be incorporated. Signals from the sound module can be analyzed by the computer module to determine the position of the sound sources. Consider a concert with IPS cymatics, wherein one or more IPS is positioned on or above the stage. Setup is initiated and test sounds are transmitted. IPS locates the relative positions of the sound sources. The operator selects which sound sources IPS should react to or ignore. IPS can be set to continually update the relative position of moving sound sources. IPS scans the topography of the projection zone. A singer sings a steady note. IPS projects a sound visualization that appears as a standing wave pattern on the ground walls and ceiling. Another singer sings a different note. The visualization portrays the volume, and pitch. The singers sing harmonic notes together and the complex interactions of constructive and destructive interference is apparent in the visualization. A drummer strikes a drum and a visualization like a pressure wave moves at the speed of sound across the ground. Observers far from the stage see the visualized pressure wave moving toward them before they hear the sound of the drumbeat. At the same instant the visualization reaches them they hear the sound. IPS can use exclusion zones to avoid shining onto people, or adjust beam intensity and shape to be eye-safe so that crowd scanning is acceptable. IPS can project volumetric cymatic effects by several means. For example, a thin reflective sheet is suspended in a concert hall. IPS scans the reflective sheet and adjusts the beam shaping optics to project a large volume beam at the sheet. The beam is reflected from the sheet into the cymatic display space that is filled with smoke or some other diffusing substance. As sound hits the reflective sheet the sheet is shaped by the sound waves and in turn shapes the reflected beam. Concave shapes in the reflective sheet will focus portions of the reflected beam and convex shapes will defocus other portions. As a result, viewers will see brighter and darker shapes moving through the cymatic display space that correspond to the sounds they hear.

According to aspects of the present invention, one or more IPS is capable of producing a photoacoustic effect that is highly directional. This capability is hereinafter referred to as “directed photoacoustics” or “directed photoacoustic effect”. FIG. 12 illustrates the directional photoacoustic effect. A laser source E1 produces a laser beam E2 that passes through beam steering optics E3. When the laser beam E2 hits the photoacoustic surface E5, a sound wave E7 is produced and propagates outward from the laser spot E6. If the beam steering optics are modulated to sweep the laser beam E2 through a beam path E4, the laser spot E6 will move across the photoacoustic surface E5. The laser spot E6 moving across the photoacoustic surface E5 causes a series of sound waves E7 to propagate outward. The series of sound waves E7 combine into a wave front E8 that propagates along a predictable wave front direction E9. This directional photoacoustic effect shares some of the principles of phased array transmitters. In this example, every atom excited by the laser spot E6 becomes a transmitter in a large passive array. The radial component of the wave front direction E9 can be steered by changing the beam path E4. The elevation component of the wave front direction E9 can be steered by changing the sweep speed along the beam path E4. The volume of the wave front E8 may be controlled by the laser beam power. The laser beam power may be modulated by microphone input to transmit speech, tones or other sounds.

Sophisticated beam patterns may produce any variety of wave front shapes including steerable columnated sound beams, steerable focal points, standing sound waves, twisting sound waves. Most common surfaces have photoacoustic properties. Darker surfaces have a stronger photoacoustic effect than lighter surfaces. Visible light, invisible light, and other sources of radiant energy can be used to produce this directional photoacoustic effect. Directional photoacoustic effect has application in telecommunication, holography, projected directional speakers, projected microphones, noise cancelation, acoustic levitation, acoustic tweezers, acoustic spanners. Projectors could project video only, sound only, or video and sound with no speakers required. The sound produced can be steered to selected areas or observers. Holograms can be projected with steerable soundtrack. It has been demonstrated that an invisible light beam focused on a window or surface can cause a reflection that is modulated by sound near the surface. The modulated reflection can be detected and converted back into sound allowing remote listening from great distances. With directed photoacoustic effect the communication could be two way. The projected beam could be modulated by microphone input. The beam propagates through a window into a room and creates a projected speaker on a surface that converts the beam signal back into sound. The projected speaker can be made to sound in all directions or be steered to a particular observer. Sound in the room would modulate the reflection. The reflection can be remotely detected and turned back into sound allowing two-way directed communication. Military could covertly communicate with no receiver equipment required.

In other embodiments, one or more IPS can project geometry and images onto sports fields while avoiding projection onto players and other protected objects. For example, IPS projects the line of scrimmage and first down line onto a football field. The ball may have reflective or emissive properties that make it identifiable to the IPS. If the ball crosses specified boundaries, the projected boundary lines change color and strobe to aid the referees.

It should be understood that, within the context of the present invention, reference objects are objects such as reflectors, lights, objects of known geometry and position, that are easily detected by IPS. Reference objects may be placed to define points of interest such as projection origin or projection boundaries. The geometry and position of reference objects generally aid in determining projector position and orientation. Mirrors may be utilized to expand the IPS field of view. Mirrors may be flat, curved, convex, or concave.

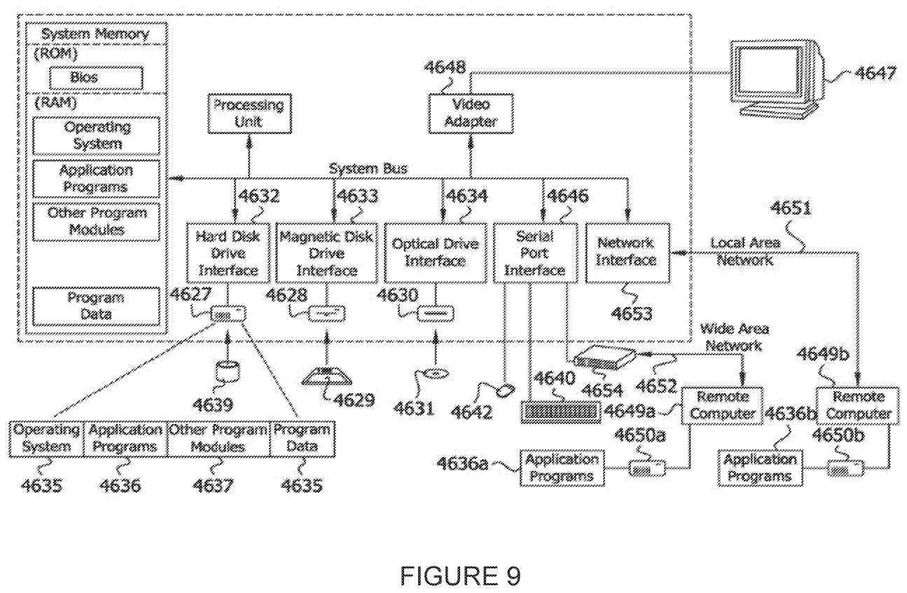

With reference to FIG. 9 an exemplary system for implementing aspects of the invention includes a general-purpose computing device in the form of a conventional computer 4320, including a processing unit 4321, a system memory 4322, and a system bus 4323 that couples various system components including the system memory 4322 to the processing unit 4321. The system bus 4323 may be any of several types of bus structures including a memory bus or memory controller, a peripheral bus, and a local bus using any of a variety of bus architectures. The system memory includes read only memory (ROM) 4324 and random-access memory (RAM) 4325. A basic input/output system (BIOS) 4326, containing the basic routines that help transfer information between elements within the computer 20, such as during start-up, may be stored in ROM 4324.

The computer 4320 may also include a magnetic hard disk drive 4327 for reading from and writing to a magnetic hard disk 4339, a magnetic disk drive 4328 for reading from or writing to a removable magnetic disk 4329, and an optical disk drive 4330 for reading from or writing to removable optical disk 4331 such as a CD-ROM or other optical media. The magnetic hard disk drive 4327, magnetic disk drive 4328, and optical disk drive 30 are connected to the system bus 4323 by a hard disk drive interface 4332, a magnetic disk drive-interface 33, and an optical drive interface 4334, respectively. The drives and their associated computer-readable media provide nonvolatile storage of computer-executable instructions, data structures, program modules, and other data for the computer 4320. Although the exemplary environment described herein employs a magnetic hard disk 4339, a removable magnetic disk 4329, and a removable optical disk 4331, other types of computer readable media for storing data can be used, including magnetic cassettes, flash memory cards, digital video disks, Bernoulli cartridges, RAMs, ROMs, and the like.

Program code means comprising one or more program modules may be stored on the hard disk 4339, magnetic disk 4329, optical disk 4331, ROM 4324, and/or RAM 4325, including an operating system 4335, one or more application programs 4336, other program modules 4337, and program data 4338. A user may enter commands and information into the computer 4320 through keyboard 4340, pointing device 4342, or other input devices (not shown), such as a microphone, joystick, game pad, satellite dish, scanner, or the like. These and other input devices are often connected to the processing unit 4321 through a serial port interface 4346 coupled to system bus 4323. Alternatively, the input devices may be connected by other interfaces, such as a parallel port, a game port, or a universal serial bus (USB). A monitor 4347 or another display device is also connected to system bus 4323 via an interface, such as video adapter 4348. In addition to the monitor, personal computers typically include other peripheral output devices (not shown), such as speakers and printers.

The computer 4320 may operate in a networked environment using logical connections to one or more remote computers, such as remote computers 4349a and 4349b. Remote computers 4349a and 4349b may each be another personal computer, a server, a router, a network PC, a peer device or other common network node, and typically include many or all of the elements described above relative to the computer 4320, although only memory storage devices 4350a and 4350b and their associated application programs 36a and 36b have been illustrated in FIG. 1A. The logical connections depicted in FIG. 9 include a local area network (LAN) 4351 and a wide area network (WAN) 4352 that are presented here by way of example and not limitation. Such networking environments are commonplace in office-wide or enterprise-wide computer networks, intranets and the Internet.

When used in a LAN networking environment, the computer 4320 is connected to the local network 4351 through a network interface or adapter 4353. When used in a WAN networking environment, the computer 4320 may include a modem 4354, a wireless link, or other means for establishing communications over the wide area network 4352, such as the Internet. The modem 4354, which may be internal or external, is connected to the system bus 4323 via the serial port interface 4346. In a networked environment, program modules depicted relative to the computer 4320, or portions thereof, may be stored in the remote memory storage device. It will be appreciated that the network connections shown are exemplary and other means of establishing communications over wide area network 4352 may be used.

One or more aspects of the invention may be embodied in computer-executable instructions (i.e., software), such as a software object, routine or function (collectively referred to herein as a software) stored in system memory 4324 or non-volatile memory 4335 as application programs 4336, program modules 4337, and/or program data 4338. The software may alternatively be stored remotely, such as on remote computer 4349a and 4349b with remote application programs 4336b. Generally, program modules include routines, programs, objects, components, data structures, etc. that perform particular tasks or implement particular abstract data types when executed by a processor in a computer or other device. The computer executable instructions may be stored on a computer readable medium such as a hard disk 4327, optical disk 4330, solid state memory, RAM 4325, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like.

A programming interface (or more simply, interface) may be viewed as any mechanism, process, or protocol for enabling one or more segment(s) of code to communicate with or access the functionality provided by one or more other segment(s) of code. Alternatively, a programming interface may be viewed as one or more mechanism(s), method(s), function call(s), module(s), object(s), etc. of a component of a system capable of communicative coupling to one or more mechanism(s), method(s), function call(s), module(s), etc. of other component(s). The term “segment of code” in the preceding sentence is intended to include one or more instructions or lines of code, and includes, e.g., code modules, objects, subroutines, functions, and so on, regardless of the terminology applied or whether the code segments are separately compiled, or whether the code segments are provided as source, intermediate, or object code, whether the code segments are utilized in a run-time system or process, or whether they are located on the same or different machines or distributed across multiple machines, or whether the functionality represented by the segments of code are implemented wholly in software, wholly in hardware, or a combination of hardware and software. By way of example, and not limitation, terms such as application programming interface (API), entry point, method, function, subroutine, remote procedure call, and component object model (COM) interface, are encompassed within the definition of programming interface.

Aspects of such a programming interface may include the method whereby the first code segment transmits information (where “information” is used in its broadest sense and includes data, commands, requests, etc.) to the second code segment; the method whereby the second code segment receives the information; and the structure, sequence, syntax, organization, schema, timing and content of the information. In this regard, the underlying transport medium itself may be unimportant to the operation of the interface, whether the medium be wired or wireless, or a combination of both, as long as the information is transported in the manner defined by the interface. In certain situations, information may not be passed in one or both directions in the conventional sense, as the information transfer may be either via another mechanism (e.g. information placed in a buffer, file, etc. separate from information flow between the code segments) or non-existent, as when one code segment simply accesses functionality performed by a second code segment. Any (or all) of these aspects may be important in a given situation, e.g., depending on whether the code segments are part of a system in a loosely coupled or tightly coupled configuration, and so this list should be considered illustrative and non-limiting.

This notion of a programming interface is known to those skilled in the art and is clear from the provided detailed description. Some illustrative implementations of a programming interface may also include factoring, redefinition, inline coding, divorce, rewriting, to name a few. There are, however, other ways to implement a programming interface, and, unless expressly excluded, these, too, are intended to be encompassed by the claims set forth at the end of this specification.

Embodiments within the scope of the present invention also include computer-readable media and computer-readable storage media for carrying or having computer-executable instructions or data structures stored thereon. Such computer-readable media can be any available media that can be accessed by a general purpose or special purpose computer. By way of example, and not limitation, computer-readable storage media may comprise RAM, ROM, EEPROM, CD-ROM or other optical disk storage, magnetic disk storage, or other magnetic storage devices, e.g., USB drives, SSD drives, etc., or any other medium that can be used to carry or store desired program code means in the form of computer-executable instructions or data structures and that can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such a connection is properly termed a computer-readable medium. Combinations of the above should also be included within the scope of computer-readable media. Computer-executable instructions comprise, for example, instructions and data which cause a general-purpose computer, special purpose computer, or special purpose processing device to perform a certain function or group of functions.

While various user functionality is described above, these examples are merely illustrative of various aspects of the present invention and is not intended as an exhaustive or exclusive list of features and functionality of the invention. Other features and functionality, while not expressively described, may be provided and/or utilized to effect and/or execute the various displays, functionality, data storage, etc.

According to aspects of the present invention, embodiments of present invention may include one or more special purpose or general-purpose computers and/or computer processors including a variety of computer hardware. Embodiments may further include one or more computer-readable storage media having stored thereon firmware instructions that the computer and/or computer processor executes to operate the device as described below. In one or more embodiments, the computer and/or computer processor are located inside the apparatus, while in other embodiments, the computer and/or computer processor are located outside or external to the apparatus.

One of ordinary skill in the pertinent arts will recognize that, while various aspects of the present invention are illustrated in the FIGURES as separate elements, one or more of the elements may be combined, merged, omitted, or otherwise modified without departing from the scope of the present invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1: A computerized system for interactively projecting images into a projection zone, said system comprising:
at least one light projecting device;
at least one computing device, said computing device being in operative communication with said at least one light projecting device for transmitting control signals to said at least one light projecting device, said computing device including one or more computer processors; and
one or more computer-readable storage media having stored thereon computer-processor executable instructions, said instructions comprising instructions for controlling said at least one light projecting device to project one or more pre-determined images into the projection zone.

2: The computerized system of claim 1, said light projecting device comprises a projecting device having high power optical output.

3: The computerized system of claim 1, said system further including at least one scanning device, wherein said computing device is in operative communication with said at least one scanning device, wherein said instructions further comprise instructions for:
receiving data from the at least one scanning device; and
controlling said at least one light projecting device to project one or more pre-determined images into the projection zone with at least one correction factor based on said received data.

4: The computerized system of claim 3, wherein said at least one scanning device includes an imaging device comprising at least one of a light detecting and ranging (LIDAR) device and a camera, wherein said received data includes topographical indicators from said imaging device, wherein said correction factor includes one or more adjustments to said one or more projected images based on the topographical indicators.

5: The computerized system of claim 3, wherein said received data indicates the presence of at least one object in the projection zone, wherein said instructions further comprise instructions for:
determining, from said receiving data, at least one protected object zone for the at least one object in the projection zone; and
controlling said at least one light projecting device to project one or more pre-determined images into the projection zone with at least one correction factor based on said at least one protected object zone.

6: The computerized system of claim 5, said light projecting device comprising a high-power projecting device, wherein said correction factor avoids projecting high-powered light onto said at least one object, wherein said high-powered light remains visible regardless of ambient light conditions.

7: The computerized system of claim 5, wherein an optical power output of said light projecting device is selectively modulated based on the properties of said object in the projection zone to prevent at least one of eye damage, skin damage, and material damage.

8: The computerized system of claim 4, wherein an optical power output and a beam shape of said light projecting device are selectively modulated based one or more reflective properties of said projection zone to enable consistent image visibility across said projection zone, regardless of any inconsistent surfaces in said projection zone, and to avoid unintended specular reflections.

9: The computerized system of claim 5, wherein said instructions further comprise instructions for projecting an illuminated image around said at least one protected object zone.

10: The computerized system of claim 1, where said controlling said at least one light projecting device includes controlling an intensity of the projected image.

11: The computerized system of claim 1, said instructions further comprising instructions for controlling said at least one light projecting device to sanitize at least one of a surface and a space in the projection zone.

12: The computerized system of claim 1, said instructions further comprising instructions for controlling said at least one light projecting device to generate at least one light barrier in the projection zone, wherein said light barrier acts as a barrier to one or more pathogens.

13: The computerized system of claim 1, said instructions further comprising instructions for generating at least one projection and at least one of directional sound and omnidirectional sound.

14: The computerized system of claim 13, wherein said directional sound comprises at least one of speech, music, or other sounds.

15: The computerized system of claim 13, wherein said omnidirectional sound comprises at least one of speech, music, or other sounds.

16: The computerized system of claim 4, said scanning device having an optical range enabling long-distance scans from an angle of intercept regardless of ambient light conditions and weather conditions, said topographical indicators and correction factors being of a number and density suitable to ensure accurate geometric correction of images being projected onto environments with major and minor variations in topography.
